(19) United States
(12) Patent Application Publication
(10) Pub. No.: US 2024/0315718 A1
(43) Pub. Date: Sep. 26, 2024

(54) MEDICAL INSTRUMENT AND METHOD FOR OPERATING A MEDICAL INSTRUMENT

(71) Applicant: BLAZEJEWSKI MEDI-TECH GMBH, Sexau (DE)

(72) Inventor: Reinhold BLAZEJEWSKI, Gutach (DE)

(21) Appl. No.: 18/619,259

(22) Filed: Mar. 28, 2024

Related U.S. Application Data
(63) Continuation of application No. PCT/DE2022/100718, filed on Sep. 27, 2022.

(30) Foreign Application Priority Data
Sep. 28, 2021 (DE) DE102021125161.3

(51) Int. Cl. A61B 17/29; A61B 17/00; A61B 17/3201; A61B 17/3211; A61B 17/34; A61B 90/00

(52) U.S. Cl. CPC A61B 17/2909; A61B 17/00234; A61B 17/3201; A61B 17/3211; A61B 17/34; A61B 90/361; A61B 90/37; A61B 2017/00199; A61B 2017/00738; A61B 2017/2903; A61B 2017/2911; A61B 2017/2929; A61B 2017/2945; A61B 2217/007

(57) ABSTRACT

A medical instrument for processing structures of a human or animal body by holding, gripping, clamping, separating, or cutting a structure or bringing together several structures, the medical instrument comprising an instrument head configured to come into contact with the structure of the human or animal body and to process the structure; an elongated instrument shaft including a proximal end and a distal end, wherein the instrument head is arranged at the distal end; at least one actuating device arranged at the proximal end of the instrument shaft and configured to actuate the medical instrument; an imaging device arranged on or in the instrument shaft and including at least one image sensor and at least one objective lens, wherein the instrument head and the imaging device are arranged on the instrument shaft fixed relative to each other in the longitudinal direction of the instrument shaft.

### RELATED APPLICATIONS

This application is a continuation of International patent application PCT/DE2022/100718 filed on Sep. 27, 2022 claiming priority from German patent application DE 10 2021 125 161.3 filed on Sep. 28, 2021, both of which are incorporated in their entirety by this reference.

### FIELD OF THE INVENTION

The invention relates to a medical instrument for processing on structures of a human or animal body.

### BACKGROUND OF THE INVENTION

Medical instruments are inserted into a human or animal body for examination or treatment. The examination or treatment often takes place in cavities or recesses. This is especially true for minimally invasive surgery. Endoscopes are often used to view the area into which the medical instruments are inserted. They have an endoscope shaft that contains an imaging system. The distal end of the endoscope shaft is inserted into the human or animal body. The endoscope is usually provided with an illumination system to illuminate the structure to be examined or treated in the cavity. The light generated by a light source is usually transmitted through optical fibers to the distal end of the endoscope shaft. The imaging system is used to record the information contained in the light reflected from a structure as an image. Image converter chips, such as CMOS or CCD, are often used as image sensors. The image sensor, also known as an imager or image converter, converts an optical image into electrical signals that are then displayed on a screen or monitor. The vision system can be provided with an image sensor to generate a two-dimensional image of the structure being inspected. Alternatively, the imaging system may be provided with two image sensors to generate a three-dimensional image of the structure being processed. Such endoscopes are also known as 3D endoscopes or stereo endoscopes.

Endoscopes can be provided with a working channel into which a medical instrument is inserted. These include gripping or cutting tools for obtaining tissue samples or removing fine soft tissue or cartilage, cannulas for injections, or wire electrodes for coagulation with an electric current. The working channel has an opening at the distal end of the endoscope shaft. The medical instrument with its instrument head emerges from this opening. The medical instrument can be moved longitudinally in the working channel to guide it to the structure to be treated in the cavity. Using the imaging system, a structure in the body cavity can be treated with the medical instrument under visual control. The instrument is movable relative to the endoscope to align the instrument relative to the structure to be treated and relative to the field of view displayed by the endoscope.

A disadvantage is that the medical instrument can be moved into the field of view of the endoscope's imaging system in such a way that it obscures the user's view of the structure being treated. The user is then unable to see the portion of the medical instrument that is in contact with the structure of the human or animal body being processed. This makes the procedure much more difficult and also poses a risk to the person or animal because visual inspection is impossible or at least more difficult. In principle, the user can, at least temporarily, align the shaft of the endoscope with the imaging device on the one hand and the medical instrument guided in the working channel of the endoscope on the other hand in such a way that the view of the structure to be processed is unobstructed. Furthermore, this alignment cannot be maintained over a longer period of time during the use of the medical instrument, since the medical instrument is moved during the processing of the structure, whereby its alignment relative to the endoscope shaft and thus to the imaging system changes.

### BRIEF SUMMARY OF THE INVENTION

The object of the invention is to provide a medical instrument that ensures visual inspection of an area to be processed where the instrument comes into contact with a human or animal body, even if the area to be processed is located in a cavity or in a non-visible recess of the human or animal body.

The object is achieved by a medical instrument for processing structures of a human or animal body by holding, gripping, clamping, separating, or cutting a structure or bringing together several structures, the medical instrument including an instrument head configured to come into contact with the structure of the human or animal body and to process the structure; an elongated instrument shaft including a proximal end and a distal end, wherein the instrument head is arranged at the distal end; at least one actuating device arranged at the proximal end of the instrument shaft and configured to actuate the medical instrument; an imaging device arranged on or in the instrument shaft and including at least one image sensor and at least one objective lens, wherein the instrument head and the imaging device are arranged on the instrument shaft fixed relative to each other in the longitudinal direction of the instrument shaft, and wherein the objective lens and a tip of the instrument head facing away from the instrument shaft are permanently aligned relative to each other so that the imaging device always generates an image of the tip of the instrument head.

The instrument includes an instrument head which comes into contact with the structure of the human or animal body and processes the structure, an elongated instrument shaft which having a proximal end and a distal end, whereby the instrument head is arranged at the distal end, an actuating device at the proximal end of the instrument shaft with which the instrument is actuated, and an imaging device which is arranged on the instrument shaft and has at least one image sensor and at least one objective lens. The instrument head and the imaging device are arranged on the instrument shaft such that they cannot be displaced in the longitudinal direction of the instrument shaft. The objective lens of the imaging device and the tip of the instrument head facing away from the instrument shaft are permanently aligned relative to each other in such a way that the imaging device always generates an image of this tip of the instrument head. Thus, the portion of the instrument head that interacts with a structure of the human or animal body at the site of use of the medical instrument and acts on that structure in the desired manner always remains in the field of view of the imaging device, even if the user moves the instrument to manipulate the structure. The portion of the structure that the instrument is acting on during processing is also in the imaging device's field of view. This prevents the instrument shaft from blocking the view of the structure being processed.

The imaging device has an optical axis. Specifically, the objective lens has an optical axis. This extends as a geometric straight line from the objective lens in the direction of the instrument head. The imaging device is oriented relative to the instrument head in such a way that the optical axis is permanently and unchangeably aligned with the portion of the instrument head with which the processing is performed. This means that the optical axis of the objective lens, which is a geometric straight line, intersects the instrument head. As a result, the imaging device is permanently positioned and aligned so that it always captures an image of at least the surface of the instrument head that touches the structure of the human or animal body during processing, as well as the area surrounding that surface. The image captured by the imaging device thus always and reliably shows the portion of the instrument head that comes into contact with the structure to be treated and its surroundings. The environment includes, in particular, the structure to be treated, provided that the instrument is close enough to the structure at the point of use. The imaging device shall be oriented so that the instrument head does not obstruct the view of the area where the instrument head contacts the structure. This area is typically located at the tip of the instrument head. The user can therefore bring the instrument close to the structure to be processed and perform the processing of the structure under reliable visual control. The imaging device provides the user with a permanent and reliable view of the area where the instrument is attached to the human or animal body.

The imaging device is adjusted once to image the essential area of the instrument head. This adjustment is made when the instrument is manufactured. It is then maintained and never changed. There is no need for adjustment or setting by the user. The image produced by the imaging device always shows the surface of the instrument head that is intended to come into contact with the structure of the human or animal body during processing, regardless of the position and orientation of the medical instrument and regardless of the location and user of the instrument.

The instrument head is specifically designed for processing and may include one or two blades or cutting edges, a clamp, a gripping tool, a cannula, or wire electrodes. Processing includes Holding, gripping, clamping, separating, or cutting the structure, or bringing multiple structures together. For example, if the instrument head is designed as a gripping tool, the gripping tool must be opened and closed during processing. For this purpose, the gripping tool is provided with gripping jaws or cutting edges, for example. One or two jaws or blades are movably arranged on the instrument head so that the medical instrument designed as a gripping tool can be opened and closed. Each jaw or blade is part of a lever. The two levers are articulated to each other. Either both jaws or blades can be moved to open and close the instrument. Alternatively, one jaw can be rigidly connected to the instrument head and only the second jaw can be articulated to the instrument head.

The instrument shaft and actuating device are used to guide the instrument head to the point of use and move it there to perform the desired procedure on the structure. This includes guiding the instrument head to and from the structure to be treated, and moving the instrument head during the treatment if the specific type of treatment requires it. If the instrument has at least one moving part mounted on the instrument head, such as forceps, the actuation device is also used to move that part relative to the instrument shaft. For example, the actuation device may have at least one handle. The handle can be moved manually or with a manipulator.

The image is transmitted from the imaging device to a data processing device by means of a signal line or by radio and finally to an image output device. This can be a display device, in particular a monitor or VR goggles.

The medical instrument may be combined with an endoscope. For this purpose, it can be inserted into the working channel of an endoscope. In this case, images are generated by the imaging device of the medical instrument and by the imaging device of the endoscope. This can be useful when the structure to be processed with the medical instrument and its surroundings are first inspected with the endoscope, and then the structure is processed with the medical instrument. The imaging device of the endoscope provides an overview of the entire structure and its surroundings, while the imaging device of the medical instrument specifically generates an image of only the portion of the instrument and the portion of the structure that interact during processing.

Alternatively, the medical instrument can be used without an endoscope. This is the case when the imaging device of the instrument is sufficient to provide the user with a visual impression of the site of application and a second imaging device can be dispensed with. The medical device may be introduced into a human or animal body cavity by means of a trocar.

The medical device can be directly operated by a physician or healthcare professional. This is done by manually operating the medical instrument's actuation device.

Alternatively, the medical instrument can be inserted into a robotic surgical system. In this case, the medical instrument is guided by a manipulator. The manipulator is coupled to the medical instrument's actuation device. In this case, the manipulator can be controlled using the image data obtained with the imaging device arranged on the instrument.

According to an advantageous embodiment of the invention, the instrument head is angled or curved at least in sections with respect to a longitudinal axis of the instrument shaft. This aligns the instrument head in the direction of the objective lens. This ensures that images of the instrument shaft are coupled into the imaging device with the objective lens.

According to a further advantageous embodiment of the invention, the objective lens and the image sensor are arranged on an image sensor holding device, wherein the image sensor holding device is accommodated on the instrument shaft and aligns the objective lens in the direction of the tip of the instrument head facing away from the instrument shaft.

According to a further advantageous embodiment of the invention, the instrument shaft accommodates an illumination which illuminates the instrument head. For example, a light source may be disposed in the instrument shaft for this purpose. The light source can be located at the distal end of the instrument shaft or at the proximal end of the instrument shaft or in between. If the light source is located at a distance from the distal end of the instrument shaft, the light from the light source can be directed to the distal end of the instrument shaft via a light guide. At the distal end of the instrument shaft, the light is emitted toward the instrument head. A light-emitting diode is particularly suitable as a light source. The light source is preferably powered by a power source that also powers the imaging device.

According to a further advantageous embodiment of the invention, the light source is rigid and stationary on the instrument. Thus, like the imaging device, it does not change its orientation and position on the instrument.

According to another advantageous embodiment of the invention, at least one irrigation channel is arranged in the instrument shaft. An irrigation medium exits through the irrigation channel at the distal end of the instrument shaft. The irrigation medium can then be selectively discharged again, for which purpose a second irrigation channel can be provided. Rinsing with a rinsing medium improves the view of the structure to be treated at the point of use of the medical instrument, for example, if blood is leaking from the structure to be examined.

According to a further advantageous embodiment of the invention, the image sensor is designed to convert optical images into electrical signals. For example, it may be a semiconductor device, in particular a CCD or CMOS. The imaging device is provided with an electrical signal line which feeds the electrical signals to a data processing device and/or an image output device. The data processing device processes the electrical signal in such a way that it can be displayed with the display device and the user receives an impression of the location of the medical instrument by means of an image output device designed as a display device and an image displayed there. If the image output device is a display device, it can be, for example, a monitor or VR goggles.

According to a further advantageous embodiment of the invention, the signal line is accommodated in the instrument shaft of the medical instrument.

According to a further advantageous embodiment of the invention, the instrument shaft is provided with a longitudinally extending channel having a first opening at the proximal end of the instrument shaft and a second opening at the distal end. The imaging device is inserted into the channel through the first opening and advanced to the distal end of the instrument shaft. At the distal end of the instrument shaft, the channel has a transparent second opening. In addition, the instrument shaft is provided with a locking device that locks the imaging device in the channel and connects it immovably to the instrument shaft. By releasing the lock, the imaging device can be detached from the instrument and removed. This allows the imaging device to be inserted into different instruments. This means that only one imaging device is needed to operate several different medical instruments. In addition, the imaging device can be detached from the medical instrument for sterilization and reuse of the medical instrument. The instrument is then sterilized without the imaging device. This applies to reusable or multiple use medical instruments. Alternatively, the medical instrument can be designed for single use, while the imaging device can be reused. In this case, it is also advantageous if the imaging device can be removed from the medical instrument.

According to a further advantageous embodiment of the invention, the imaging device is provided with an interface via which image data is output by radio to a data processing device. In this case, a cable-based signal line can be dispensed with. This facilitates the handling of the medical device. The data processing device is used to process the data generated by the imaging device and to display it to a user on a display device.

According to a further advantageous embodiment of the invention, the medical instrument is provided with an energy storage device. This can be in the form of a battery or a rechargeable battery. In this case, a cable for supplying power to the imaging device can be dispensed with. This facilitates the handling of the medical device at its place of use.

According to a further advantageous embodiment of the invention, the imaging device is designed as a stereo imaging device, also known as a 3D imaging device. With a suitable viewing device, the user can be provided with a three-dimensional image of the instrument head and its surroundings. In this case, the user can more easily orient himself at the location where the instrument will be used and determine the distance between the structure to be treated and the instrument head. This can be done either qualitatively with respect to a reference point, or quantitatively if the imaging device is calibrated accordingly. In this case, special viewing devices are required that separately display image data for the left eye and image data for the right eye.

According to a further advantageous embodiment of the invention, the 3D imaging device comprises a left image sensor and a right image sensor. This produces two images which are combined in a display device to form a three-dimensional image. Alternatively, an image sensor may be used having a first number of pixels for a left image and a second number of pixels for a right image.

According to a further advantageous embodiment of the invention, the focus of the imaging device is adjusted such that the tip of the instrument head facing away from the instrument shaft is in the focus of the imaging device. The focus is set by the objective lens and, if necessary, additional lenses of the imaging device. The focus of the objective lens is selected to match the distance between the objective lens and the tip of the instrument head.

According to a further advantageous embodiment of the invention, the instrument is designed as a cutting tool. In particular, the instrument can be configured as a scalpel. It can also be designed as a pair of scissors. Alternatively, the instrument can be designed as a razor. A shaver is used to remove fine soft tissue or cartilage and is mainly used in arthroscopy.

According to another advantageous embodiment of the invention, the instrument is designed as a gripping tool. For example, the instrument may be a pair of forceps. It can also be a pair of pliers.

According to another advantageous embodiment of the invention, the instrument is designed as a holding tool. For example, it can comprise a clamp.

According to another advantageous embodiment of the invention, the instrument is designed as a trocar. A trocar is used to create a sharp or blunt access to a body cavity, such as an abdominal or thoracic cavity. This access is kept open by a tube. The imaging device on the trocar facilitates positioning at the surgical site.

According to a further advantageous embodiment of the invention, the instrument is formed as a cannula.

According to a further advantageous embodiment of the invention, the instrument comprises at least one wire electrode for coagulation with electric current.

According to a further advantageous embodiment of the invention, the instrument is designed for single use. It can therefore be disposed of after use and does not need to be sterilized for reuse.

According to a further advantageous embodiment of the invention, the instrument is reusable. It is designed to be sterilizable. In particular, it is suitable for treatment in an autoclave.

According to a further advantageous embodiment of the invention, the medical instrument is provided with an aperture, which blanks out the instrument shaft and/or a portion of the instrument head facing the instrument shaft from the images recorded by the imaging device. Thus, the user's visual impression of the displayed images is not affected by the instrument shaft or the portion of the instrument head facing the instrument shaft.

According to the method of the invention for operating a medical instrument, the images produced with the imaging device are processed and visualized on an image output device. The images are processed in such a way that the instrument shaft and/or a portion of the instrument head facing the instrument shaft is blanked out in the images displayed on the image output device. Therefore, the instrument shaft or the portion of the instrument head facing the instrument shaft is not visible in the images displayed by the image output device. The user's attention is therefore not drawn to the instrument shaft or the portion of the instrument head immediately adjacent thereto, but only to the distal tip of the instrument head facing away from the instrument shaft, which interacts with a structure to be treated and treats it in a targeted manner. Other advantages and advantageous embodiments are set forth in the claims.

### BRIEF DESCRIPTION OF THE DRAWINGS

The invention is subsequently described based on embodiments with reference to drawing figures, wherein:

FIG. 1 shows a first embodiment of a medical instrument in perspective view;

FIG. 2 shows a proximal end of the instrument shaft and the actuating device of the instrument of FIG. 1;

FIG. 3 shows a distal end of the instrument shaft and the instrument head of the instrument shown in FIG. 1;

FIG. 4 shows an image of the instrument head of the instrument of FIG. 1 generated by the imaging device and displayed on a visualization device;

FIG. 5 shows an image of FIG. 4 with partially obscured instrument head;

FIG. 6 shows an image of an instrument head of a second embodiment of a medical instrument generated with an imaging device and displayed on a visualization device;

FIG. 7 shows an image of FIG. 6 with partially obscured instrument head;

FIG. 8 shows a third embodiment of a medical instrument in perspective view;

FIG. 9 shows a proximal end of the instrument shaft and the actuating device of the instrument according to FIG. 8, wherein the imaging device is accommodated and locked in the instrument shaft;

FIG. 10 shows a proximal end of the instrument shaft according to FIG. 9 with the imaging device unlocked and partially released from the instrument shaft;

FIG. 11 shows an instrument head and distal end of the instrument shaft of the medical device of FIG. 8 in plan view;

FIG. 12 shows a perspective view of the instrument head and distal end of the instrument shaft of the medical device shown in FIG. 8; and

FIG. 13 shows a front view of the instrument head and distal end of the instrument shaft of the medical device shown in FIG. 8.

### DETAILED DESCRIPTION OF THE INVENTION

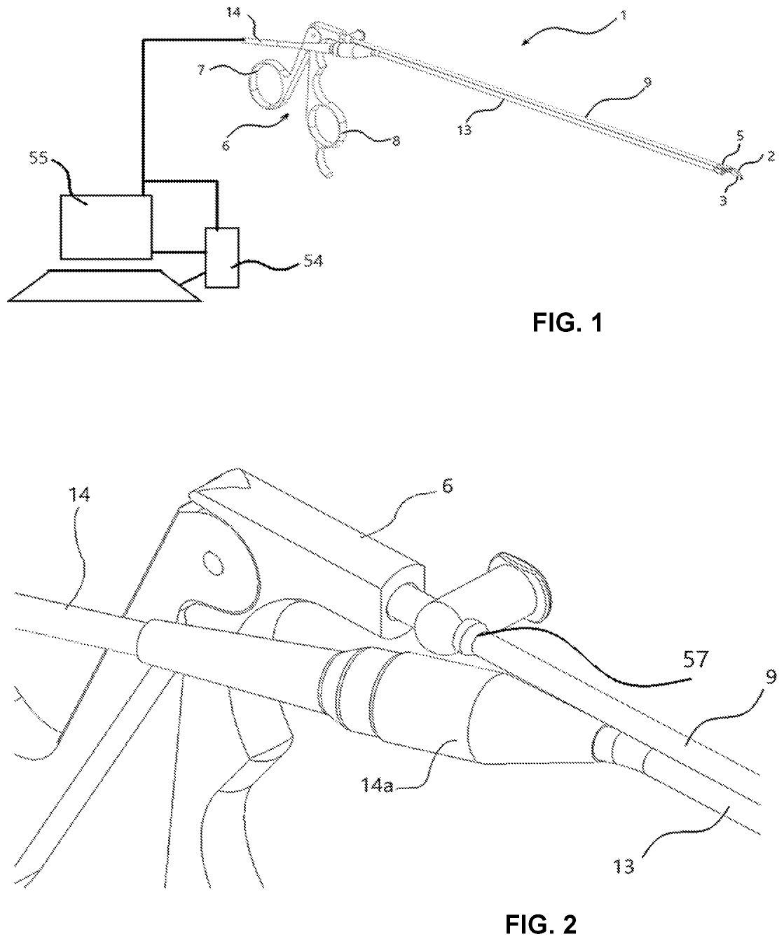

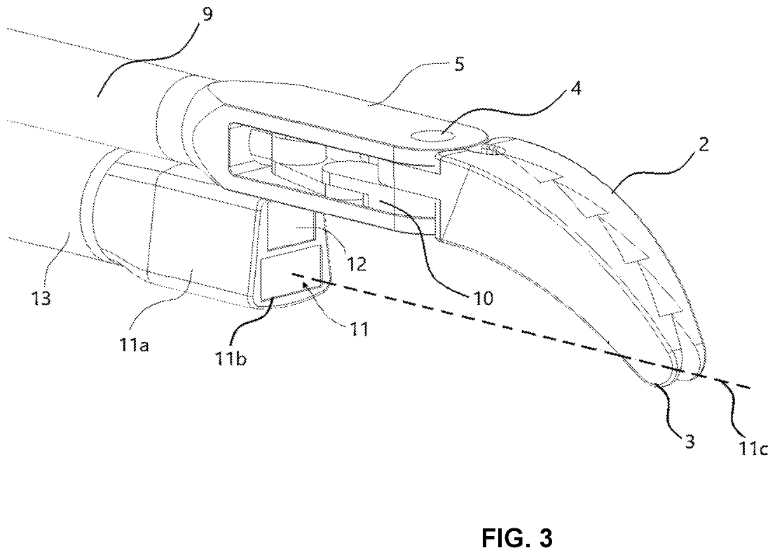

FIGS. 1 to 5 show an example of a first embodiment of a medical instrument 1. The instrument includes a pair of forceps designed as a two-blade tool and provided with two gripping jaws 2, 3. The two gripping jaws 2, 3 are designed as levers. The two gripping jaws 2, 3 have a serrated surface on the sides facing each other. The two gripping jaws are connected by a joint 4 and are arranged on an instrument head 5. The instrument 1 has an elongated instrument shaft 9. At a first end, referred to as the distal end, the instrument head 5 is arranged on the instrument shaft 9. At a second end, which is referred to as the proximal end of the instrument shaft, a first opening 57 and two ring-shaped handle parts 7, 8 are arranged. These handle parts 6, 7 are part of an actuating device 6 by which the medical instrument 1 can be operated manually. A first handle part 7 is movably connected to the instrument shaft 9 via a joint. A second handle part 8 is rigidly connected to the instrument shaft 9. The two gripping jaws 2, 3 are moved by moving the two handle parts 7, 8. The gripping jaws 2, 3 and the handle parts 7, 8 each have an open position and a closed position. The movement of the handle parts 7, 8 is transmitted to the gripping jaws 2, 3 by a mechanism. The mechanism is partially located in the instrument shaft 9. The two gripping jaws 2, 3, the joint 4 and parts of the mechanism 10 are located on the instrument head 5. In addition, an imaging device 11 and an illumination 12 are arranged in a fixed position on the instrument shaft 9. The imaging device 11 comprises an image sensor and an objective lens 11b arranged in an imaging housing 11a. The image sensor is completely enclosed by the imaging housing 11a and included in the imaging device 11. The objective lens 11b and the image sensor are arranged on an image sensor holding device 56 arranged on the instrument shaft 9. The objective lens 11b is attached to an opening window of the imaging housing 11a. In the illustration according to FIG. 3, the viewer looks at this opening window provided with the objective lens 11b. The imaging device 11 and the illumination 12 are arranged and adjusted on the instrument shaft 9 in such a way that the objective lens 11b and the illumination are permanently aligned with the two ends of the gripping jaws 2, 3 of the instrument head 5 facing away from the instrument shaft 5. The imaging device thus captures images of the two gripping jaws 2, 3 in their open position, in their closed position and in all positions of the gripping jaws 2, 3 in between. If a structure, e.g. a tissue, is picked up by the two gripping jaws 2, 3 of the instrument head 5, this structure is also recognizable in the images recorded by the imaging device.

The objective lens 11b has an optical axis 11c which extends as a geometric straight line in the direction of the gripping jaws 2, 3. The instrument head 5 has a curvature in the direction of this optical axis 11c. The gripping jaws 2, 3 are curved for this purpose. The optical axis 11c intersects the two gripping jaws 2, 3 at their ends facing away from the instrument shaft 9, which are also referred to as the distal ends of the two gripping jaws 2, 3. This makes it possible, in particular, to optically detect the distal ends of the two gripping jaws 2, 3.

The imaging device 11 comprises an image sensor which converts optical signals into electrical signals. The instrument shaft 9 is provided with a channel 13. An electrical signal line of the imaging device 11 and a supply line for the illumination 12 run in this channel 13. The signal line and the supply line are brought together at the proximal end in a cable 14 with a cable housing 14a. This cable 14 can be connected to a visualization device on which the images generated by the imaging device 11 are displayed. The visualization device is not shown in the drawing.

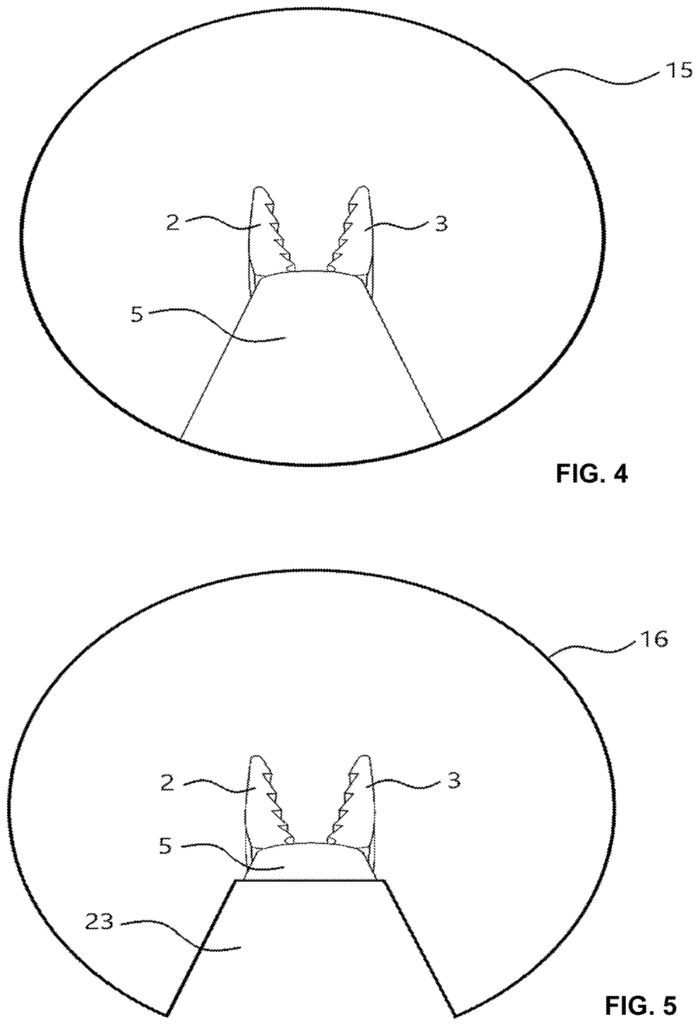

FIG. 4 shows an image 15 of the two gripping jaws 2, 3 in the open position generated by the imaging device 11. This image generated by the imaging device 11 is displayed on a visualization device not shown in the drawing. The portion of the instrument head 5 on which the two gripping jaws 2, 3 are movably mounted is also visible in the image 15.

When a user moves the instrument 1 towards a structure at its place of use and first opens the two gripping jaws 2, 3, picks up the structure at least in sections and then closes the gripping jaws 2, 3 again, the gripping jaws 2, 3 and a structure arranged in the gripping jaws are always visible to the user with the imaging device 11. Since the position of the joint 4 of the gripping jaws 2, 3 and the imaging device 11 is fixed and does not change when the instrument is actuated, the processing area of the two gripping jaws is always and permanently visible with the imaging device. The viewing area shown in FIG. 15 is not obscured by parts of the instrument or the imaging device.

FIG. 5 shows another image 16 generated by the imaging device of the gripping jaws 2, 3 of the medical instrument. In this image 16, the portion of the instrument head 5 facing the instrument shaft is obscured. For this purpose, the area 23 is cut out of the image 16. This is done by means of image processing. Hiding the instrument head makes it easier for the user to work, as he is less distracted by the section of the instrument head 5 facing the instrument shaft.

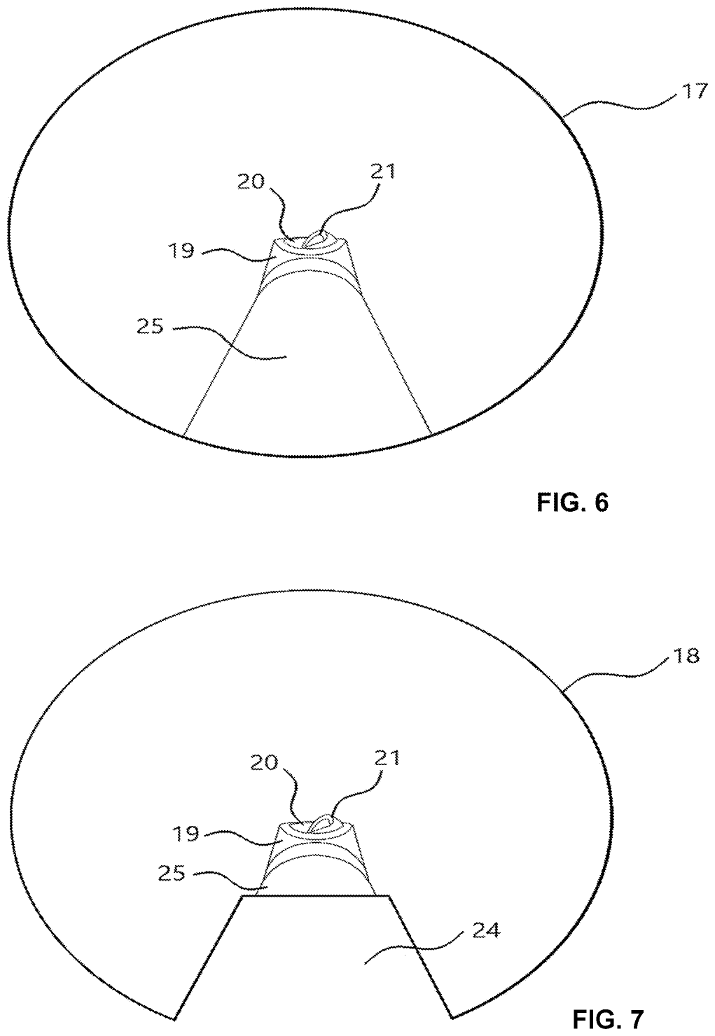

FIGS. 6 and 7 show images 17, 18 of a second embodiment of a medical instrument. These images 17, 18 have been generated with an imaging device corresponding to images 15, 16 arranged on the second embodiment example of the medical instrument. The medical instrument is a shaver for removing fine soft tissue or cartilage. The shaver comprises a housing 19 with an opening 20 on an instrument head 25. A knife 21 is rotatably mounted in the housing 19. The medical instrument with the shaver can be constructed in the same manner as the first embodiment example of a medical instrument in FIGS. 1 to 5, except for the housing 19 with the opening 20 and the knife 21. The housing 19 and the knife 21 are arranged on the instrument head 25 which is partially visible in FIG. 17 according to FIG. 6. In FIG. 18 according to FIG. 7, the portion of the instrument head 25 facing an instrument shaft is hidden. For this purpose, the area 24 is cut out of the image 18 by means of image processing.

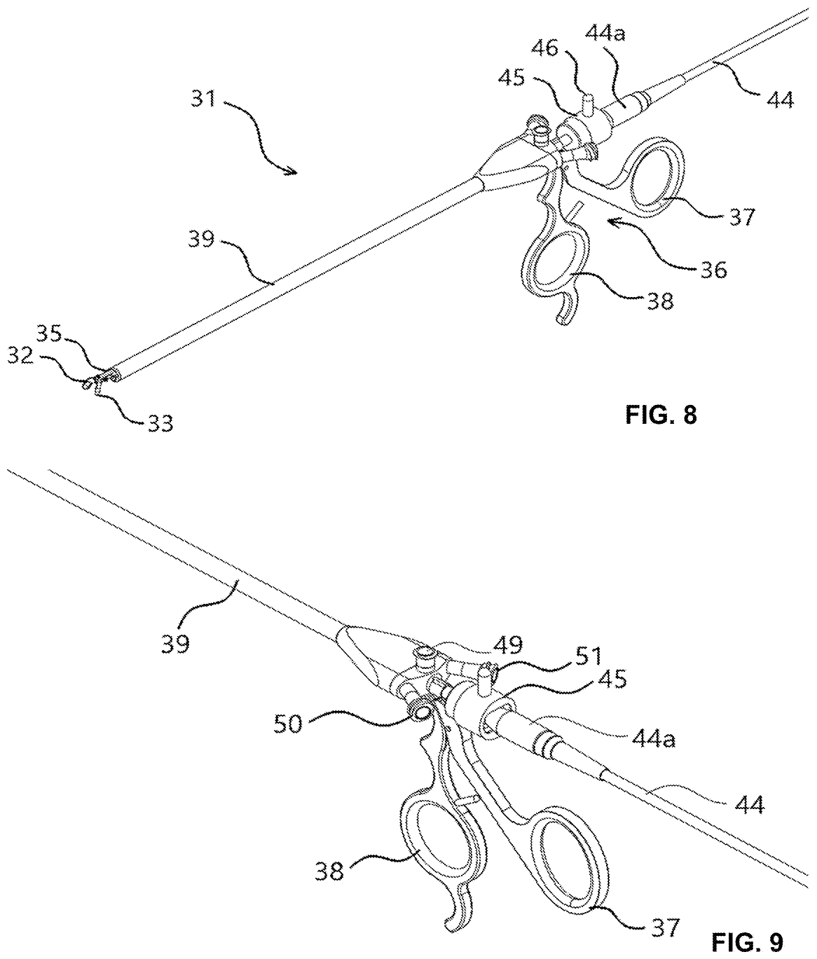

A third embodiment of a medical instrument 31 is shown in FIGS. 8 through 13. This is a spoon forceps. It has two gripping jaws 32, 33 which are designed as levers and are arranged on an instrument head 35 so that they can rotate about a joint 34. The two gripping jaws 32, 33 are part of the instrument head 35. They are curved in the direction of the optical axes 41c of the imaging device 41. The optical axes 41c are geometrical straight lines.

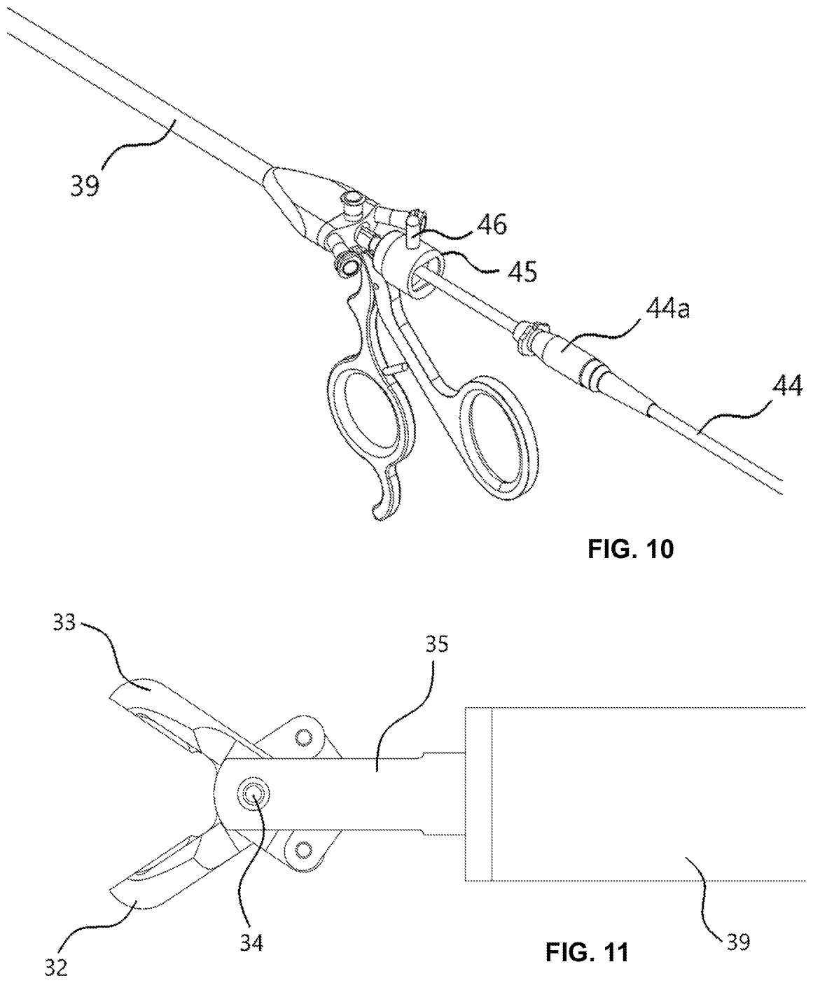

In contrast to the first embodiment example according to FIGS. 1 to 5, the two gripping jaws of the medical instrument 31 according to FIGS. 8 to 13 each have a recess on the sides facing each other. According to the first embodiment example, the medical instrument 31 according to the third embodiment example has an elongated instrument shaft 39, at the distal end of which the instrument head 35 and at the proximal end of which an actuating device 36 with two gripping parts 37, 38 are arranged. The two gripping jaws 32, 33 are moved by the two handle parts 37, 38. In contrast to the medical instrument 1 according to FIGS. 1 to 5, the imaging device 41 in the medical instrument 31 according to FIGS. 8 to 13 is detachably accommodated in the instrument shaft 39. For this purpose, the instrument shaft 39 has a channel 43 which is provided with an opening at its distal end and at its proximal end. A locking device 45 with a locking pin 46 is arranged at the opening at the proximal end. The imaging device 41 has a signal line accommodated in a cable 44 having a cable housing 44a. The imaging device 41 is inserted with this cable 44 into the channel of the instrument shaft 39 and advanced in the direction of the distal end of the instrument shaft 39 until the objective lens 41a, 41b are at their predetermined end position at the distal end of the instrument shaft 39. Once this end position is reached, the imaging device 41 is locked to the instrument shaft 39. For this purpose, the locking pin 46 is brought to its locking position on the locking device 45. In this locking position, the locking pin 46 engages with the cable housing 44a. This prevents the imaging device 41 from moving in the longitudinal direction of the instrument shaft 39. FIGS. 8 and 9 show the imaging device 41 in its locked end position in the instrument shaft 39. FIG. 10 shows the imaging device 41 in an unlocked position, partially withdrawn from the instrument shaft 39.

Another difference between the first and third embodiments is that the medical instrument 31 of FIGS. 8 through 13 is provided with irrigation channels 47, 48. These have spigots 49, 50, 51 at the proximal end of the instrument shaft 39 and openings 52, 53 at the distal end of the instrument shaft 39. Lines not shown in the drawing can be connected to the spigots 49, 50, 51. They serve to supply and discharge an irrigation medium, which exits through one of the openings 52 and is sucked out through the other opening 53.

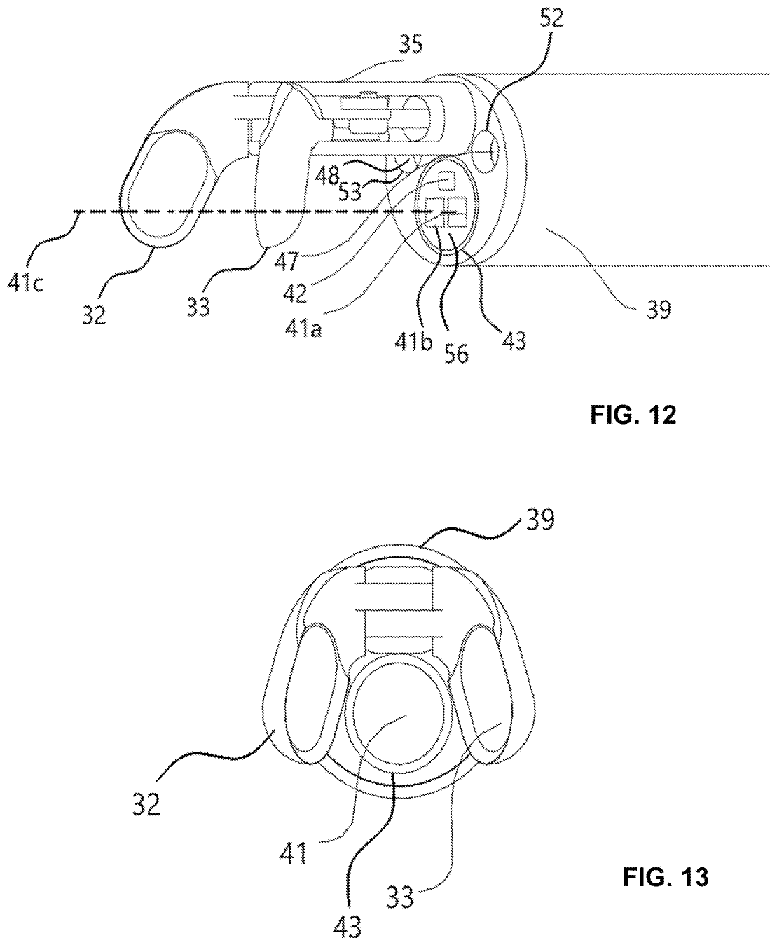

A further difference between the first and third embodiments is that the imaging device 41 of the medical instrument 31 according to FIGS. 8 to 13 is a 3D imaging device. It has a left image channel and a right image channel. An objective lens 41a, 41b and an image sensor are associated with each image channel. The image sensors are located in the instrument shaft 39 and are therefore not visible in the drawing. The imaging device is also provided with an illumination 42. Each objective lens 41a, 41b is associated with an optical axis 41c corresponding to a geometric straight line. The optical axes 41c of the two objective lenses 41a, 41b may be parallel to each other or oriented at an angle other than 0° to each other. In the medical instrument 31, the optical axes are parallel to each other. In the illustration shown in FIG. 12, only one of the two optical axes 41c is visible. The other optical axis is located behind the optical axis 41c in the illustration so that it is hidden.

Images produced by the imaging device 41 must be displayed with a viewing device capable of three-dimensional display. For this purpose, the images of the left image channel must be displayed for the left eye of a user and the images of the right image channel must be displayed for the right eye.

All of the features of the invention may be essential to the invention both individually and in any combination.

### REFERENCE NUMERALS AND DESIGNATIONS

1 Medical instrument
2 Gripping jaw
3 Gripping jaw
4 Joint
5 Instrument head
6 Actuating device
7 Handle part
8 Handle part
9 Instrument shaft
10 Mechanism
11 Imaging Device, Image Sensor
11a Imaging Housing
11b Objective lens
11c Imaging device optical axis
12 Illumination
13 Duct
14 Cable, Electrical, Signal Line
14a Cable Housing
15 Image
16 Image
17 Image
18 Image
19 Housing
20 Opening
21 Knife
23 Area cut out of the image
24 Area cut out of the image
25 Instrument head
31 Medical instrument
32 Gripping jaw
33 Gripping jaw
34 Joint
35 Instrument head
36 Actuating device
37 Handle part
38 Handle part
39 Instrument Shaft
41 Imaging Device
41a Objective lens
41b Objective lens
41c Optical axis
42 Illumination
43 Channel
44 Cable
44a Cable Housing
45 Locking Device
46 Locking Pin
47 Irrigation Channel
48 Flush Channel
49 Connector
50 Spigot
51 Spigot
52 Opening
53 Opening
54 data processing device
55 image output device
56 image sensor holding device
57 first opening

What is claimed is:

1. A medical instrument for processing structures of a human or animal body by holding, gripping, clamping, separating, or cutting a structure or bringing together several structures, the medical instrument comprising:
an instrument head configured to come into contact with the structure of the human or animal body and to process the structure;
an elongated instrument shaft including a proximal end and a distal end, wherein the instrument head is arranged at the distal end;
at least one actuating device arranged at the proximal end of the instrument shaft and configured to actuate the medical instrument;
an imaging device arranged on or in the instrument shaft and including at least one image sensor and at least one objective lens,
wherein the instrument head and the imaging device are arranged on the instrument shaft fixed relative to each other in the longitudinal direction of the instrument shaft, and
wherein the objective lens and a tip of the instrument head facing away from the instrument shaft are permanently aligned relative to each other so that the imaging device always generates an image of the tip of the instrument head.

2. The medical instrument according to claim 1, wherein the instrument head is curved or angled at least in sections with respect to a longitudinal axis of the instrument shaft and is thereby aligned in a direction of the objective lens.

3. The medical instrument according to claim 1,
wherein the objective lens and the image sensor are arranged on an image sensor holding device,
wherein the image sensor holding device is arranged on the instrument shaft, and
wherein the objective lens is aligned in a direction towards the tip of the instrument head that faces away from the instrument shaft.

4. The medical instrument according to claim 1, further comprising: an illumination which illuminates the instrument head is received in the instrument shaft.

5. The medical instrument according to claim 1, further comprising: at least one irrigation channel arranged in the instrument shaft.

6. The medical instrument according to claim 1,
wherein the at least one image sensor is configured to convert optical images into electrical signals, and
wherein the imaging device is provided with an electrical signal line which feeds the electrical signals to a data processing device and/or an image output device.

7. The medical instrument according to claim 6, wherein the signal line is arranged in the instrument shaft.

8. The medical instrument according to claim 1,
wherein the instrument shaft includes a longitudinally extending channel including a first opening at the proximal end of the instrument shaft, through which the imaging device is inserted into the longitudinally extending channel and advanced to the distal end of the instrument shaft,
wherein the instrument shaft includes a transparent second opening at the distal end of the instrument shaft, and
wherein the instrument shaft includes a locking device which locks the imaging device arranged in the longitudinally extending channel and locks the imaging device in the instrument shaft.

9. The medical instrument according to claim 1, wherein the imaging device is configured as a 3D imaging device.

10. The medical instrument according to claim 1, further comprising: an aperture which removes the instrument shaft and/or a portion of the instrument head facing the instrument shaft from images recorded by the imaging device.

11. The medical instrument according to claim 1, configured as a scalpel.

12. The medical instrument according to claim 1, configured as a shaver.

13. The medical instrument according to claim 1, configured as a forceps.

14. The medical instrument according to claim 1, configured as a pair of scissors.

15. The medical instrument according to claim 1, configured as a clamp.

16. The medical instrument according to claim 1, configured as a tweezer.

17. The medical instrument according to claim 1, configured as a trocar.

18. The medical instrument according to claim 1, further comprising: at least one wire electrode.

19. A method for operating the medical instrument according to claim 1, the method comprising:
producing the images with the imaging device; and
processing the images and visualizing the images on an image output device,
wherein the images are processed so that the instrument shaft and/or a portion of the instrument head facing the instrument shaft is removed from the images and not displayed on the image output device.
